(19) United States
(12) Patent Application Publication
(10) Pub. No.: US 2008/0114343 A1
(43) Pub. Date: May 15, 2008

(54) Skeletal muscle revascularization

(76) Inventors: James Lowe, Hillsborough, NC (US); Gary Allen, Celebration, FL (US); Elbert Tzeng, Irvine, CA (US); Richard Lanigan, San Juan Capistrano, CA (US); John McIntyre, Rancho Santa Margarita, CA (US)

Correspondence Address: STETINA BRUNDA GARRED & BRUCKER, 75 ENTERPRISE, SUITE 250, ALISO VIEJO, CA, 92656, US

(21) Appl. No.: 11/598,476

(22) Filed: Nov. 13, 2006

(51) Int. Cl. A61B 18/18; A61B 17/34

(52) U.S. Cl. 606/15; 606/16; 606/185

(57) ABSTRACT

Methods for the treatment of ischemic limbs. An energy source, which will typically be a laser energy source deployed through an optic fiber, is deployed subcutaneously to a site of ischemic skeletal muscle. At the site, a plurality of channels are preferably systematically formed with an appropriate amount of activated energy, which in turn can facilitate the restoration of blood flow through new blood vessel formation. Optionally, a needle or cannula can be deployed in conjunction with the energy source to help facilitate the formation of channel formation and/or deliver a therapeutic agent at the sight of channel formation. A sheath may also be utilized to house the optic fiber and deliver a therapeutic agent.

### CROSS-REFERENCE TO RELATED APPLICATIONS

Not Applicable

### STATEMENT RE: FEDERALLY SPONSORED RESEARCH/DEVELOPMENT

Not Applicable

### BACKGROUND

This invention is for the treatment of ischemic limbs in patients who are otherwise not revascularizable, and more particularly for the treatment of ischemic limbs where open or percutaneous revascularization are not viable alternative medical procedures.

Ischemia in skeletal muscle is due to a lack of adequate blood flow. This occurs due. to an occlusion in the blood vessel that supplies blood to the region and prevents adequate blood supply from reaching the distal extremity. One of the symptoms of ischemia in these regions is pain in the extremities, known as claudication. One treatment of ischemic skeletal muscle is to perform a bypass procedure in which another vessel or a synthetic vessel (e.g., a PTFE or woven polyester material) is sewn proximal to the vessel and distal to the occlusion in the artery. This procedure restores blood flow distal to the occlusion and provides a renewed supply of blood to the ischemic skeletal muscle. Another treatment is the use of stents in the occluded vessel to reopen the vessel to adequate blood flow. Surgery on the vessel can also be performed to strip out the vessel occlusion in a procedure called endarterectomy.

Each of these procedures, however, has particular limitations. In a bypass procedure, the effectiveness of the procedure can be limited by the size of the distal blood vessel, the size (diameter and length) of the synthetic vessel, and the distal runoff. Because of these limitations, the graft used to bypass may become occluded itself. In stenting and angioplasty, only short occlusions can be bypassed because of the need to perform the procedure percutaneously and to pass a guidewire distal to the occlusion. In addition, in these procedures, the diseased vessel remains in place and is prone to reocclusion over time. Endarterectomies are similarly limited and tend to result in a weak vessel that is prone to re-occlusion. Most of the limitations of limb ischemia treatment occur in the distal regions in which vessel size is small, and in below the knee procedures the vessel size is most limited and thus most susceptible. In addition, when bypassing a vessel below the knee, the bypass vessel must also be flexible enough to withstand the various motions. of the knee without severely kinking.

In severe cases, blood flow in the ischemic skeletal muscle cannot be adequately restored, and as a consequence, the extremity must often times be amputated. This is almost always viewed as a procedure of last resort, but appropriate where no other alternatives are available. Accordingly, there is a substantial need in the art that is operative to restore blood flow in ischemic skeletal muscle that overcomes the foregoing limitations, and preferably can promote angiogenesis and vessel collateralization to a degree sufficient to provide adequate blood supply to a distal extremity.

### BRIEF SUMMARY

The present invention specifically addresses and alleviates the above-identified deficiencies in the art. In this regard, the current invention is for a method to treat ischemic skeletal muscle; and in particular for methods to treat patients with claudication and/or critical limb ischerinia, especially when existing medical procedures cannot or should not be performed. To that end, the present invention expressly contemplates the application of techniques associated with transmyocardial revascularization (TMR) but adapts such procedure for applications involving skeletal muscle. In this regard, TMR is currently a known method to treat ischemic cardiac muscle. Using TMR; energy is used to create small channels across the ischemic left ventricle, and could potentially be used to treat other regions of the heart. Generally, laser energy. typically provided by either a CO2; excimer, or Holmium: YAG laser source is used to create these channels. Initially it was thought that these channels would remain open to the left ventricle and the blood from the LV would provide a constant source of blood for the ischemic myocardium. However, it has been learned that these channels do not remain patent long term and that other mechanisms are responsible for the relief of angina pain and reduction of ischenmia. These long term benefits are due to angiogenesis and neovasculanzation (the creation of new blood vessels).

While laser energy sources are currently used for TMR therapy and are deemed suitable for the methods disclosed herein, other energy sources could potentially be used in the present invention directed to skeletal muscle. These other sources include RF energy, ultrasound energy, mechanical means, and cryogenic energy. In all cases, the tissue surrounding the created channel is disrupted, which activates the body's natural healing process and stimulates an angiogenic response. This angiogenic response ultimately results in new blood vessels which supply blood to the tissue and relieve the ischemia present.

The current invention proposes to use the foregoing technique to: treat the ischemic skeletal muscle in a similar manner., As contemplated, a small tunnel is made subcutaneously through which an energy source will be deployed, preferably through an optic fiber, at a site of ischemic skeletal muscle. The energy source will be operative to form channels within the ischemic skeletal muscle at a designated site. Channels can be created with any car the above mentioned energy sources, however, it would be convenient and easy to use if the energy source can be delivered. through a flexible tube, and especially through a fiber optic for laser energy delivery.

In a further optional refinement of the present invention, it is contemplated that the aforementioned procedures may be used in combination with a needle or cannula, the latter being operative to facilitate channel formation in combination with the energy source, as well as serve as a conduit for delivering a therapeutic agent at the sight at which the channel is formed. To that end, it is contemplated that the needle may be used in close proximity with the energy source, which will typically be deployed through an optic fiber, to thus facilitate the discrete formation of channels, as well as ensure accurate deployment of the desired therapeutic agent. In an alternative refinement, the optical fiber can be contained within any suitable covering or sheath. The sheath can have an additional lumen through which a therapeutic agent can be delivered within the channel created. Delivery in this way can be done while the optical fiber is still in place within the channel, as it is being retracted, or after the fiber has been retracted. Furthermore, the channel created can be filled with solid, foam, or gel therapeutic material or impregnated biocompatible material (e.g., collagen sponge material or collagen sponge material impregnated with a therapeutic agent).

Creating channels with an appropriate amount of activated tissue around the channel can stimulate the angiogenic response in the ischemic skeletal muscle. This response can result in new blood vessel formation, the resumption of blood flow to the ischemic skeletal muscle, and the relief of symptoms due to severe ischemia. The procedure likewise avoids the various drawbacks of the other aforementioned procedures known in the art.

### BRIEF DESCRIPTION OF THE DRAWINGS

These and other features and advantages of the various embodiments disclosed herein will be better understood with respect to the following description and drawings, in which like numbers refer to like parts throughout, and in which:

FIG. 1 is a frontal perspective view of a human body depicting both upper and lower limb skeletal muscles.

FIG. 2 is a perspective view of a portion of an ischemic limb showing portions of skeletal muscle, including a cross-sectional view thereof, having a plurality of channels formed therein for facilitating the restoration of blood flow.

FIG. 3 is a partial cross-sectional view of a section of skeletal muscle having a plurality of channels being formed thereupon from an energy source.

FIG. 4 is the cross-sectional view of FIG. 3 depicting the skeletal muscle having a plurality of channels formed thereupon, the channels being operative to facilitate the restoration of blood flow to an ischemic limb.

### DETAILED DESCRIPTION

The above description is given by way of example, and not limitation. Given the above disclosure, one skilled in the art could devise variations that are within the scope and spirit of the invention disclosed herein, including various ways of treating ischemic skeletal muscles via TMR-type procedures. Further, the various features of the embodiments disclosed herein can be used alone, or in varying combinations with each other and are not intended to be limited to the specific combination described herein. Thus, the scope of the claims is not to be limited by the illustrated embodiments.

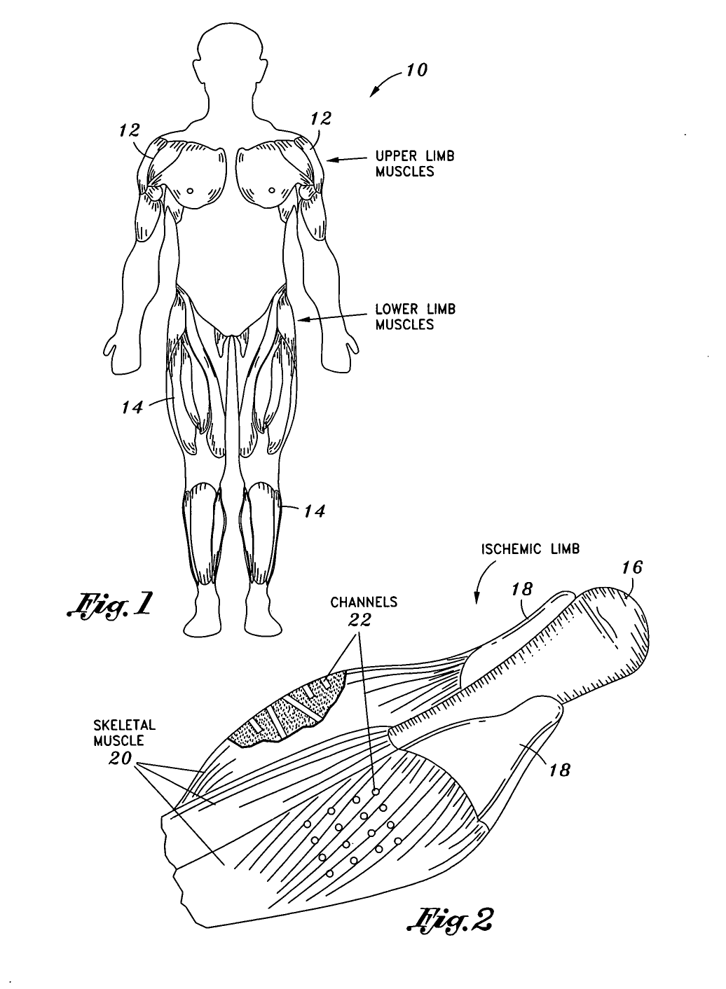

Referring now to the drawings, and initially to FIG. 1, there is shown a human FIG. 10 generally referencing the upper limb muscles 12 and lower limb muscles 14. As is readily-known to those skilled in the art, such muscles comprise skeletal muscles, namely, striated voluntary muscles, attached to bones via tendons. Such muscle is contrasted with cardiac muscle, namely, muscle of the heart (myocardium) that is composed of specialized, striated muscle cells not under voluntary control but rather responsive to the autonomic nervous system.

The present invention is directed to methods for treating ischemic skeletal muscle of the upper and lower limbs, and in particular the lower limbs that are ischemic due to diffuse and/or small vessel disease. As a consequence of such condition, an adequate blood supply is prevented from reaching the distal extremities of the limbs thus causing pain known as claudication. The present invention seeks to alleviate such condition, particularly when alternative procedures, such as open or percutaneous revascularization, are not viable options.

Instead, there is shown in FIG. 2, a novel approach for restoring blood flow to an ischemic limb via revascularization created from the formation of small channels in the ischemic skeletal muscle. In this regard, the ischemic limb will essentially be defined by bone 16 and tendons 18 attached thereto. The ischemic skeletal muscle 20 extending from tendons 18 will be identified utilizing procedures known in the art. Using procedures similar to TMR, channels 22 are formed systematically about the outer-most surface thereof via the systematic application of an energy source, discussed more fully below. As illustrated, the skeletal muscle 20 will ultimately be provided with a plurality of channels 22 that may serve to either act as bloodlines through which oxygen-rich blood may flow, and thus ultimately oxygenate skeletal muscle 20 and/or may promote angiogenesis, namely, the growth of new capillaries that will supply blood to the skeletal muscle 20.

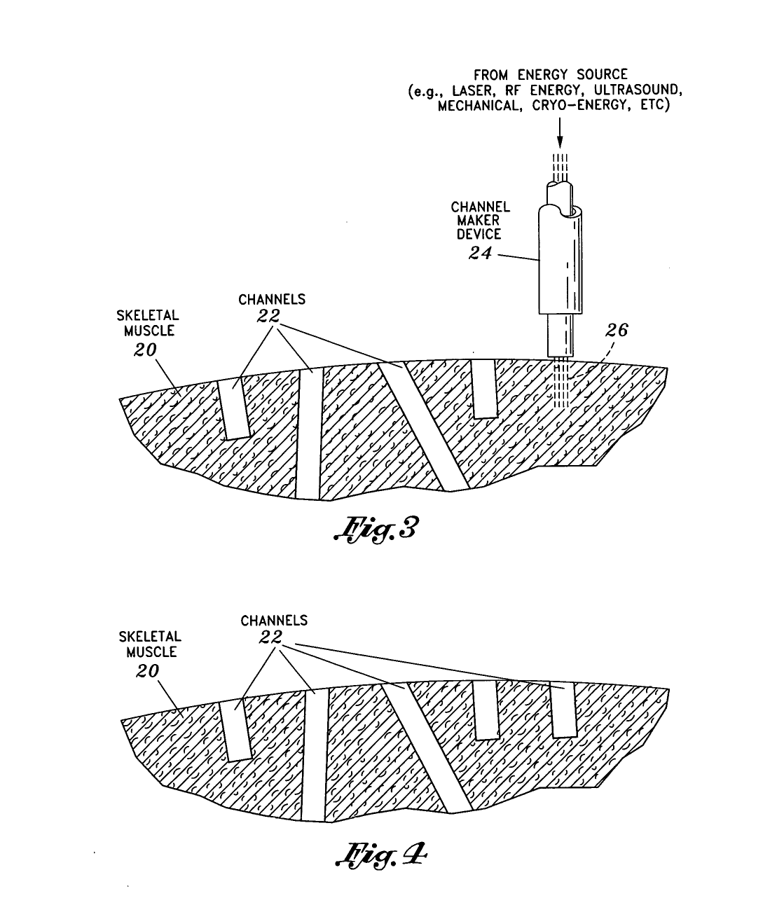

Referring now to FIGS. 3 and 4, and initially to FIG. 3, the process by which channels 22 are formed within ischemic 20 is illustrated. Initially, an energy source operative to form the channels 22 is brought into proximity with the area of ischemic skeletal muscle to be treated. To that end, it is contemplated that the energy source will be brought into proximity to the ischemic skeletal muscle 20 via a small tunnel that can be made subcutaneously. Along these lines, the formation of a subcutaneous tunnel operative to define a pathway through which the energy source can be operatively positioned relative the skeletal muscle 20 may be accomplished by a variety of surgical techniques and devices well-known to those skilled in the art, for example, as may be accomplished by tunnel devices and tunnel catheters.

Once the energy source 24 is positioned in proximity to the area of skeletal muscle to be treated 20, the energy source may be utilized to form a plurality of channels 22 within the skeletal muscle 20. As illustrated, the energy source may be operative to form channels 22 that are generally perpendicular to the outer surface of skeletal muscle 20 or, alternatively, may be angled relative the outer surface of the skeletal muscle 20. In this regard, it is contemplated that the skeletal muscle revascularization methods of the present invention can readily allow for the formation of straight and/or angled channels 22 depending on whether or not the orientation of such channel is advantageous or not. Alternatively, it is contemplated that channels 22 may not necessarily be formed to have any type of particular orientation relative the outer surface of skeletal muscle 20, as determined by the particular patient and/or procedure being performed.

With respect to the formation of the channels 22, the same will be made by the application of energy deployed through the energy source 24 whereby the emitted energy 26 will form a discrete channel. Presently, it is believed that between 2 to 3 channels per square centimeter of skeletal muscle 20 will create enough channels 22 to facilitate revascularization of the ischemic skeletal muscle 20. As will be readily understood by those skilled in the art, the depth of the channels 22, as well as the number of channels per surface area, may be selectively adjusted to the particular patient in a given condition. Moreover, it will readily be understood that the width and depth of the channels 22 may likewise be selectively chosen to attain optimal revascularization for a particular patient. Along these lines, it is contemplated that channels 22 may be formed to have a preferred width of about 1 mm and a depth ranging from about 5 millimeters to about 100 millimeters. Alternatively, the channels 22 may be formed to span the entirety of the targeted skeletal muscle 20.

With respect to the energy source utilized by the channel maker device 24, it will be readily understood by those skilled in the art that laser energy, whether CO2, excimer and/or Holmium: YAG laser sources may be readily utilized per conventional TMR procedures. Alternatively, other energy sources can be utilized, including RF energy, ultrasound energy, mechanical energy, and cryogenic energy. The present invention should further be deemed to encompass any other energy source known or later developed that would be operative to impart energy 26 sufficient to cause the formation of channels 22 within skeletal muscle 20, as shown. At present, it is believed that deployment of an energy source through a flexible tube, such as fiber optic deployment of laser energy delivery, will be most ideal given the widespread usage of fiber optics for laser energy delivery per conventional TMR procedures as well as the subcutaneous deployment of such devices. As will be readily understood by those skilled in the art, regardless of the type of energy utilized, the energy intensity utilized to form the channels 22 may be readily adjusted so as to cause the formation of channels 22 having desired dimensions, and in particular width and depth.

In a further yet optional refinement of the invention, it is contemplated that a needle or cannula (not shown) will be utilized in connection with the channel formation procedure. In this respect, it is contemplated that the needle will be situated in close proximity relative to the energy source 24, which as discussed above will preferably take the form of an optic fiber. The needle may be utilized to form an initial portion of the channel, such as a pilot hole, through which the energy source may extend the depth of the channel. To facilitate such channel formation, the needle may be positioned axially about the energy source, particularly so when the energy source is emitted through an optic fiber. Alternatively, the needle may be positioned adjacent to the energy source, and may be concurrently deployed with the energy source, or may be deployed before or after the energy source forms a respective channel 22. Still further, such needle or cannula may be useful to deploy a therapeutic agent at the sight at which a channel is formed. Such therapeutic agent, as referenced herein, should be construed broadly to encompass all types of pharmaceutical and biological materials that may have a medical purpose, and can include healing agents, genetically altered materials, stem cells or any other composition known or later developed in the art that would be useful in such applications.

In an additional, optional embodiment, it is contemplated that the optical fiber utilized to create the formation of the channels referenced herein may be contained within any suitable covering or sheath, and may further be designed to have a catheter-like structure which will be well-understood by those in the art. According to such refinement, the sheath may be provided with at least one additional lumen through which a therapeutic agent can be delivered. Along these lines, it is expressly contemplated that the therapeutic agent will be delivered within one or more of the channels created via the deployment of the energy source and/or combination of the deployed needle and energy source, as discussed above. One specific means by which the delivery of a therapeutic agent can be delivered can be achieved while the optical fiber is still in place within the channel, with the therapeutic agent being delivered through the covering or sheath as the optical fiber is being retracted or after the fiber has been completely retracted from the channel. Although it will be expressly recognized that any of a variety of therapeutic agents can be deployed within one or more of the channels created, it should expressly be recognized that any or all of the channels that have been created via the processes discussed herein can be filled with solid, foam, or gel therapeutic material or impregnated biocompatible material, such as collagen sponge material or collagen sponge material impregnated with a therapeutic agent. Accordingly, it will be readily recognized by those skilled in the art that a wide variety of therapeutic agents and the like may be readily used in conjunction with methods of the present invention.

Advantageously, it is believed that the skeletal muscle revascularization procedures of the present invention provide a highly desirable and viable alternative to other procedures known in the art for the treatment of ischemic limbs, including bypass, stenting, angioplasty and/or endarterectomy. Moreover, and perhaps most advantageous, is that the skeletal muscle revascularization procedure of the present invention provides a further procedure or measure that can be taken prior to limb amputation.

Additional modifications and improvements of the present invention may also be apparent to those of ordinary skill in the art. Thus, the particular combination of parts and steps described and illustrated herein is intended to represent only certain embodiments of the present invention, and is not intended to serve as limitations of alternative devices and methods within the spirit and scope of the invention.

What is claimed is:

1. A method for facilitating the revascularization of ischemic skeletal muscle, the method comprising the steps:
a. providing an area of ischemic skeletal muscle;
b. providing an energy source operative to emit energy through an optical fiber sufficient to cause the formation of a channel within said ischemic skeletal muscle; and
c. placing said optical fiber in proximity to the area of ischemic skeletal muscle provided in step (a) and systematically applying said energy source provided in step (b) to said area of ischemic skeletal muscle to form a plurality of channels about said area of ischemic skeletal muscle.

2. The method of claim 1 wherein in step (b), said energy source comprises laser energy.

3. The method of claim 2 wherein said laser energy is provided from a laser selected from the group consisting of Diode, excimer, Nd: YAG, and Holmium: YAG.

4. The method of claim 1 wherein in step (c) each one of said respective plurality of channels has a width of approximately one millimeter and a depth ranging from about 5 millimeters to about 100 millimeters.

5. The method of claim 1 wherein in step (c) at least one channel spans the length of said ischemic skeletal muscle.

6. The method of claim 1 wherein in step (c), said plurality of channels are formed to have a channel density from about 1 channel per cubic centimeter of ischemic skeletal muscle to about 3 channels per square centimeter of ischemic skeletal muscle.

7. The method of claim 1 wherein in step (a), said ischemic skeletal muscle consists of upper limb skeletal muscle.

8. The method of claim 1 wherein in step (a), said ischemic skeletal muscle consists of lower limb skeletal muscle.

9. The method of claim 1 wherein in step (a), said ischemic skeletal muscle occurs within a patient having vascular disease of the lower extremity arteries.

10. The method of claim 1 wherein following step (b) and prior to step (c), said method further comprises the step:
a. forming a subcutaneous tunnel defining a pathway through which said energy source may extend and become positioned in close proximity to said area of ischemic skeletal muscle provided in step (a).

11. The method of claim 1 further comprising the step:
a. providing a needle in proximity to at least one of said plurality of channels and deploying a therapeutic agent therethrough.

12. The method of claim 1 wherein following step (a) and prior to step (b), the method further comprises the step:
a. providing a needle in proximity to the area of ischemic skeletal muscle provided in step (a) and advancing the distal end of the needle into said ischemic skeletal muscle to form a pilot hole in the surface of the ischemic muscle through which the energy source can be advanced.

13. The method of claim 1 further comprising the step:
a. Deploying a therapeutic agent within the channel created.
